(19) United States
(12) Patent Application Publication
(10) Pub. No.: US 2013/0012915 A1
(43) Pub. Date: Jan. 10, 2013

(54) CONNECTING SYSTEM THROUGH WHICH FLUID FLOWS FOR USE IN MEDICINE AND MEDICAL TECHNOLOGY

(76) Inventors: Hans Juergen Hopf, Zirndorf (DE); Norbert Kassai, Oberasbach (DE); Alexander Hopf, Nuernberg (DE); Michael Hopf, Zirndorf (DE)

(21) Appl. No.: 13/574,650

(22) PCT Filed: Jan. 24, 2011
(86) PCT No.: PCT/EP2011/050922

§ 371 Date: Sep. 28, 2012

(30) Foreign Application Priority Data
Jan. 22, 2010 (DE) 20 2010 000 078.5

(51) Int. Cl. A61M 39/10; C08G 63/02

(52) U.S. Cl. 604/500; 604/535; 528/272

(57) ABSTRACT

A connecting system for components through which fluid flows for medicine and medical technology, in particular infusion or transfusion tubes, selector valves, multi-way distributors, injection equipment such as needles, accesses or the like, and combinations thereof, comprising a female tubular receptacle, which comprises an inner receiving section and an outer first fixing section, and/or a male tubular plug element for reception in the female receiving section and an outer second fixing section, which cooperates with the first fixing section for fixing the connecting system. The surfaces of the connecting system that come in contact with the fluid are produced at least in some sections from amorphous copolyester. Use of amorphous copolyester for producing a corresponding connecting system.

The present invention relates to connecting systems through which fluid flows, such as are used particularly in medicine.

Three-way stopcocks and Y-connectors for example are known from the prior art and are particularly used in medicine and medical technology. In this context, they are used particularly for infusion, in the area of artificial nutrition, for transfusions and particularly for the infusion and/or transfusion of various fluid media, and as “injection equipment” for medical and pharmaceutical devices. Three-way stopcocks are also used in medical systems that comprise multiple components, among other features. Such systems comprise among other things gravity infusion lines, pumps or pump transfusion systems, tube-fed nutrition systems, injections, combinations thereof and the like. A three-way stopcock may also be combined with several other three-way stopcocks form a selector valve assembly or “manifold”.

However, one of the disadvantages of the connecting systems through which fluid flows known from the prior art is that they are produced from plastics, the processing of which involves the use of plasticizers such as bisphenol-A. However, when the fluid systems through which fluid flows are used, it is not possible to ensure entirely that no plasticizer particles are transferred to the fluid, and it is known that these plasticizers are capable of having a harmful effect on physical health. Carcinogenic and other effects are attributed to these plasticizers.

The object of the present invention is to provide connecting systems through which fluid flows with which the possible risk of burden to the patient from undesirable accompanying substances is at least reduced.

This object is achieved by using an amorphous copolyester to manufacture the connecting systems through which fluid flows, and with corresponding connecting systems that are furnished with an appropriate material at least on the surfaces that come into contact with the fluid. Preferred variations are the object of the subordinate claims.

According to a first, particularly preferred embodiment of the present invention, an amorphous copolyester is used to produce a lockable connecting system for components used in medicine and medical technology and through which fluids flow, such as in particular infusion or transfusion tubes, selector valves, manifolds, injection equipment such as needles, accesses or the like, and combinations thereof, wherein the connecting system comprises at least one female, tubular receptacle that has an inner receiving section and a first outer fixing section, and/or a male tubular plug element for insertion in the female receiving section, and a second outer fixing section that cooperates with the first fixing section for fixing the connecting system.

According to another preferred embodiment, the invention comprises a connecting system for components through which fluids flow and that are used in the medical and medical technology fields, particularly such as infusion or transfusion tubes, selector valves, manifolds, injection equipment such as needles, accesses or the like, and combinations thereof, with a female, tubular receptacle that has an inner receiving section and a first outer fixing section, and/or a male tubular plug element for insertion in the female receiving section, and a second outer fixing section that cooperates with the first fixing section for fixing the connecting system. The system is characterized in that at least sections of at least the surfaces of the connecting system that come into contact with the fluid are made from an amorphous copolyester.

In addition, other materials are used besides the copolyester as materials for producing the connecting system according to the invention, and these are selected from a group including thermosetting and thermoplastic plastics, and particularly polyphenylene sulphide, polypropylene, poly-1-butene, polyvinyl chloride, polyvinylidene chloride, polymethyl metaacrylate, polyacrylic nitrile, polystyrene, polysulfone, polyacetal, polyvinyl alcohol, polyvinyl acetate, ionomer, fluoroplastic, polyethylene, polyamide, particularly a partially aromatic polyamide, polycarbonate, polyester, polyphenylene oxide, polysulfone, polyvinyl acetal, polyurethane, and chlorinated polyether, cellulose nitrate, cellulose acetate, cellulose ether, phenol resin, urea resin, thiourea resin, melamine resin, alkyl resin, allyl resin, silicone, polyimide, polybenzimidazole, epoxy resin, casein plastic, cross-linked polyurethane, unsaturated polyester resin, antimicrobial or antiseptic materials such as high-porosity silver, nonionically manufactured silver, silver compounds and particularly compounds that release metal ions, combinations thereof and the like.

The fluids that typically flow in the connecting system are selected from a group that includes injection solutions, infusion solutions, nutrition solutions, blood, plasma, gases, air, combinations thereof and the like.

The present invention further includes the use of the connecting system according to the invention in medicine and/or medical technology for infusing or transfusing fluids, particularly for gravity infusions, pump transfusion systems, tube-fed nutrition systems, injections, combinations thereof and the like.

In the following, the invention will be explained with reference to a preferred embodiment, and it is explicitly stated that this example also encompasses modifications and/or additions such as would be immediately evident to one skilled in the art. Furthermore, this preferred exemplary embodiment does not represent a limitation of the invention to the extent that modifications and additions fall within the scope of the present invention.

In the drawing:

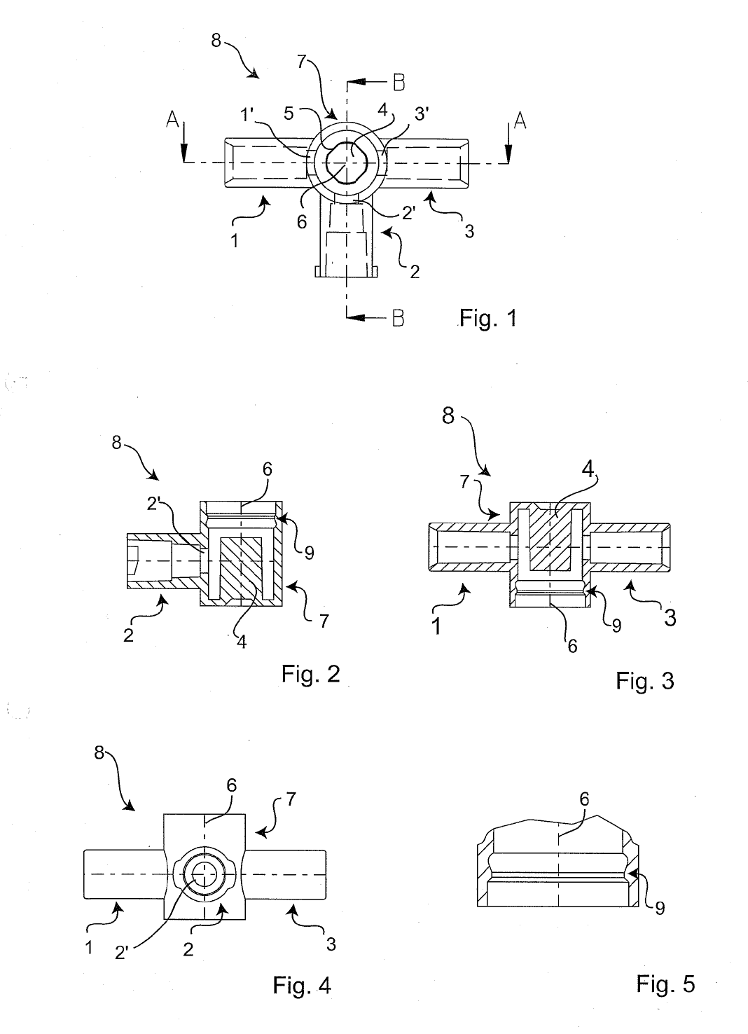

FIG. 1 is a top view of basic housing of a three-way stopcock;

FIG. 2 is a cross section through the basic housing of FIG. 1 along line of intersection B-B;

FIG. 3 is a cross section through the basic housing of FIG. 1 along line of intersection A-A;

FIG. 4 is a front view of the basic housing of FIG. 1;

FIG. 5 is a detail view of the recess that extends radially around the actuator receptacle of the basic housing;

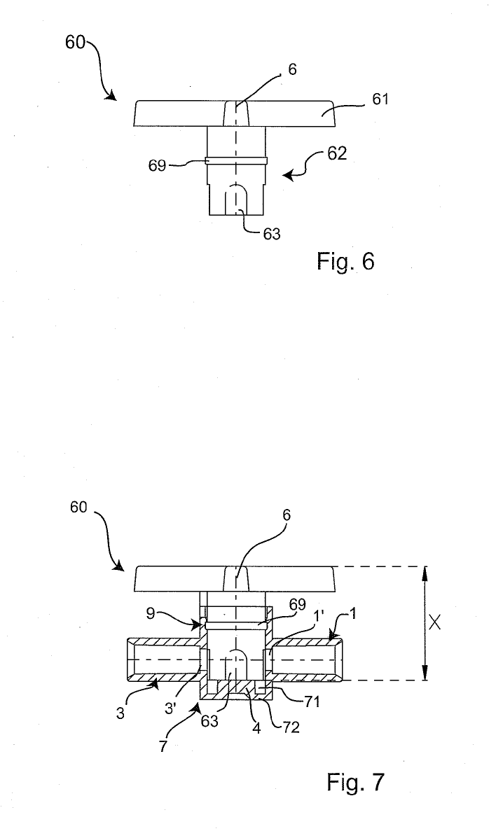

FIG. 6 is an actuator of a selector valve according to the invention;

FIG. 7 is a partial cross-section through the basic housing of FIG. 1 along line of intersection A-A with the actuator pressed in.

The embodiment of FIG. 1 shows basic housing 8 with three connection points 1 , 2, 3 and outlet openings 1′, 2′, 3′ corresponding therewith in actuator receptacle 7. A pin 4 with four flattened areas 5 is disposed about a central axis 6 in actuator receptacle 7. The dashed lines indicate the lines of intersection A-A and B-B for the cross sections in FIGS. 2 and 3.

Accordingly, FIG. 2 shows a cross section through the basic housing along line of intersection B-B. Pin 4 is disposed about central axis 7 in actuator receptacle 6. Connection point 2 is connected to the actuator receptacle, forming outlet opening 2′. The inner side of actuator receptacle 6 is furnished with a recess 9 that extends around the perimeter thereof, recess 9 being conformed in such manner that it has varying depths.

FIG. 3 shows a cross-section of the basic housing of FIG. 2 along the line of intersection marked A-A in FIG. 1. The two connection points 1 and 3 are connected to actuator receptacle 6, forming outlet openings 1′ and 3′ (FIG. 1). The inner side of actuator receptacle 6 is furnished with a recess 9 that extends around the perimeter thereof, recess 9 being conformed in such manner that it has varying depths.

According to the present embodiment, particularly the inner surfaces of the three-way stopcock are provided with an appropriate material that essentially contains no plasticizers, such as a copolyester for example. This may be achieved either by coating the surfaces accordingly, or also by producing a corresponding component made from such a material.

FIG. 4 shows a side view of the basic housing from the front corresponding to FIG. 1. Connection points 1, 2, 3 are connected to actuator receptacle 6. The corresponding outlet openings are located in the middle of these connections, such as outlet opening 2′ in connection point 2.

FIG. 5 shows a part of actuator receptacle 6 with peripheral recess 9 on the inner side thereof. The recess has varying depths, so that a series of depressions and protrusions is formed.

FIG. 6 shows an embodiment of an actuator for use in conjunction with the basic housing according to FIG. 1. The actuator comprises an operating element 61 in the form of a faucet with three control levers, which faucet is permanently connected to a hollow-cylindrical section 62. The hollow-cylindrical section is disposed concentrically about a central axis 62. Gateway-shaped outlet openings 63 are formed at the end of the hollow-cylindrical section farthest from the operating element. The number of outlet openings 63 in the actuator corresponds to the number of connection points in the basic housing. A prominent contour is provided and extends radially around the circumference of hollow-cylindrical section 62 of actuator 60.

FIG. 7 shows the connection between the basic housing (hatched area in the figure) of FIG. 1 and actuator 60 of FIG. 6 as an embodiment of the selector valve according to the invention. Actuator 60 is pressed into actuator receptacle 7 of the basic housing as far as a predetermined impression depth X. In this position prominent contour 69 of the hollow-cylindrical section of actuator 60 engages in recess 9 on the inner side of actuator receptacle 6 of the basic housing. The hollow-cylindrical section of actuator comprises the pin 4 of actuator recess 6 that is arranged about a central axis 6, thus forming a counterbearing for actuator 60. When the selector valve is open, the medium flows from one of connection points 1, 3, through the associated outlet opening in basic housing 1′, 3′, through a gateway-shaped outlet opening in actuator 63, through a gap 71 between actuator 60 and bottom 72 of actuator receptacle 6 as far as another outlet opening 63 of the actuator and out of the respective other connection point 1, 3 through the respective other outlet opening 1′, 3′ in the basic housing. A seal in the upper area is formed with a defined impression depth X of actuator 60 in the joint rim. Inside the basic housing 8, the seal with regard to the corresponding outlet openings 1, 2, 3 is assured by the conical counterbearing that is formed by pin 4. Four flattened areas 5 on pin 4 serve to ensure that the faucet moves smoothly, that is to say that actuator 60 is easily displace in actuator receptacle 7.

1.-5. (canceled)

6. A method of use of an amorphous copolyester to manufacture a lockable and/or adjustable connecting system for components for medicine and medical technology through which fluids flow, such as infusion or transfusion tubes, selector valves, manifolds, injection equipment such as needles and accesses, and combinations thereof, the method comprising the steps of:
using amorphous copolyester to manufacture a female, tubular receptacle that has an inner receiving section and a first outer fixing section; and,
using amorphous copolyester to manufacture a male tubular plug element for insertion in the female receiving section, with a second outer fixing section that cooperates with the first fixing section for fixing the connecting system.

7. A connecting system for components through which fluids flow and that are used in the medical and medical technology fields, such as infusion or transfusion tubes, selector valves, manifolds, injection equipment such as needles and accesses, and combinations thereof, comprising:
a female, tubular receptacle that has an inner receiving section and a first outer fixing section;
a male tubular plug element for insertion in the female receiving section, with a second outer fixing section that cooperates with the first fixing section for fixing the connecting system, and,
wherein at least sections of surfaces of the connecting system that come into contact with the fluid are made from an amorphous copolyester.

8. The connecting system according to claim 7, wherein other materials are used besides the amorphous copolyester are used to make the sections of surfaces of the connecting system that come into contact with the fluid, and these materials are selected from a group consisting of: thermosetting and thermoplastic plastics, polyphenylene sulphide, polypropylene, poly-1-butene, polyvinyl chloride, polyvinylidene chloride, polymethyl metaacrylate, polyacrylic nitrile, polystyrene, polysulfone, polyacetal, polyvinyl alcohol, polyvinyl acetate, ionomer, fluoroplastic, polyethylene, polyamide, particularly a partially aromatic polyamide, polycarbonate, polyester, polyphenylene oxide, polysulfone, polyvinyl acetal, polyurethane, and chlorinated polyether, cellulose nitrate, cellulose acetate, cellulose ether, phenol resin, urea resin, thiourea resin, melamine resin, alkyl resin, allyl resin, silicone, polyimide, polybenzimidazole, epoxy resin, casein plastic, cross-linked polyurethane, unsaturated polyester resin, antimicrobial or antiseptic materials, high-porosity silver, nonionically manufactured silver, silver compounds, compounds that release metal ions, and combinations thereof

9. The connecting system according to either of claim 8, wherein fluid is selected from a group that consists of: injection solutions, infusion solutions, nutrition solutions, blood, plasma, gases, air, and combinations thereof.

10. The connecting system according to either of claim 7, wherein fluid is selected from a group that consists of: injection solutions, infusion solutions, nutrition solutions, blood, plasma, gases, air, and combinations thereof.

11. A method comprising the step of:
using the connecting system according to claim 8 in medicine and/or medical technology, for infusing or transfusing fluids, gravity infusions, pump transfusion systems, tube-fed nutrition systems, injections, or combinations thereof.

12. A method comprising the step of:
using the connecting system according to claim 9 in medicine and/or medical technology, for infusing or transfusing fluids, gravity infusions, pump transfusion systems, tube-fed nutrition systems, injections, or combinations thereof.

13. A method comprising the step of:
using the connecting system according to claim 10 in medicine and/or medical technology, for infusing or transfusing fluids, gravity infusions, pump transfusion systems, tube-fed nutrition systems, injections, or combinations thereof

14. A method comprising the step of:
using the connecting system according to claim 11 in medicine and/or medical technology, for infusing or transfusing fluids, gravity infusions, pump transfusion systems, tube-fed nutrition systems, injections, or combinations thereof.
